(19) United States
(12) Patent Application Publication
(10) Pub. No.: US 2008/0107744 A1
(43) Pub. Date: May 8, 2008

(54) INJECTABLE HOLLOW TISSUE FILLER

(76) Inventor: Jack Fa-De Chu, Santa Rosa, CA (US)

Correspondence Address: JACK CHU, 5918 SUNHAWK DRIVE, SANTA ROSA, CA, 95409, US

(21) Appl. No.: 11/935,210

(22) Filed: Nov. 5, 2007

Related U.S. Application Data
(60) Provisional application No. 60/864,446, filed on Nov. 6, 2006.

(51) Int. Cl. A61K 9/14; A61F 2/02

(52) U.S. Cl. 424/489; 514/789; 623/23.73

(57) ABSTRACT

The present invention comprises a plurality of injectable hollow particulate fillers suspended in a biocompatible fluid carrier to significantly improve the clumping resistance and injectability of the composition. The hollow particulate fillers have a lower effective density and are able to suspend in the carrier without precipitation. The loss of skin volume as a result of aging, diseases, weight loss, and injury can lead to uneven skin surface (e.g. wrinkle, etc.). The uneven skin can be repaired by injecting appropriate amount of hollow fillers underneath the skin. Some cases of urinary incontinence occur when the resistance to urine flow has decreased excessively. Continence is restored by injecting the present invention to the urethra tissue to increase resistance to urine outflow. Similarly, the present invention allows for the control of gastric fluid reflux by submucosal injections of the fillers to the esophageal-gastric and gastric-pyloric junction. For patients with vesicoureteral reflux, it can be treated by injection of the present invention into patients' ureteral tissue. This invention can also be used to repair defective or inadequately functioning muscles of the anal sphincter by administering an effective amount of injectable hollow fillers into the defect or anal sinuses.

This application claims the benefit of U.S. Provisional Application No. 60/864,446, which was filed Nov. 6, 2006, the disclosure of which is incorporated herein by this reference.

### FIELD OF INVENTION

The present invention is about a new injectable hollow particulate filler used to the repair of defect or injury, to the augmentation of soft tissue, to the augmentation of a hypoplastic breast, to the augmentation of scar tissue, to the treatment of urological disorders, to the treatment of incompetent anal sphincters, to the treatment of paralysis of the vocal cords, to the treatment of vesicoureteral reflux, and to the treatment of gastric fluid reflux by endoscopical or subcutaneous injection of biocompatible hollow particular implants into the submucosal or dermal tissue.

### BACKGROUND OF THE INVENTION

The present invention addresses those aspects of designing an ideal composition for tissues that need to be repaired. The injectable composition of this invention is also suitable for the treatment of many tissue conditions such as augmentation and strengthening of tissue in patients. Other than the plastic surgery or reconstructive surgery, tissue fillers can be used to correct aphonia or dysphonia caused by paralysis of the vocal cords, to correct defect or injury, to the augmentation of hypoplastic breast, to the augmentation of scar tissue, to the treatment of urological disorders (e.g. urinary incontinence), to the treatment of incompetent anal sphincters, to the treatment of vesicoureteral reflux, and to the treatment of gastric fluid reflux by endoscopic or subcutaneous injection of biocompatible hollow particular implants into the submucosal or dermal tissue. Since the invention is closely related to the treatment of soft tissue augmentation, it will be described in details by reference hereto.

Many factors contribute to the loss of skin volume as the underlying collagen, hyaluronic acid, and elastin fibers begin to deteriorate. They can be part of aging process, diseases such as acne or cancer, weight loss, and excess exposure to sun light. This loss in skin volume creates uneven skin surface such as wrinkles, laugh lines, folds and furrows on the face.

There are several techniques to restore smoothness to the skin's surface. In the practice of plastic or reconstructive surgery, the most common non-invasive method is to build up a depressed area within the skin with a filler substance. It is injected with a fine needle below skin surface where it corrects the line or wrinkle by filling up the skin depression without leaving scar. Fillers can also be placed into the lips to create a fuller look or in the hollows of the cheeks to restore a natural appearance.

Needle injection is the preferred method to deliver fillers with minimum side effect in the target location for many physicians. The advantage for using needle is obvious. It is easy to use with a high precision and leaves no scar on the skin. With this technique, the injected filler particles have to be relatively small to pass through the needle.

A variety of biological soft tissue fillers are available for clinicians today by using several techniques. They are human and bovine collagen, hyaluronic acid, autologous fat, autologous and donor tissues. However, their effect is temporary because the body eventually breaks down the filler. Results last from several months to about a year. Patients have to be treated frequently to maintain the good results.

Several semi-permanent fillers are available in the market. Radiesse™ is composed of calcium hydroxylapatite (HA) microspheres, which are suspended in polysaccharide carrier. It has been used in the body for multiple applications including cheek and chin implants. The other semi-permanent filler is Sculptra®, which is made of synthetic polylactic acid (PLA) contained in microspheres. It is approved for restoring volume to the face of HIV patients suffering from facial lipoatrophy. The clinical results from these synthetic implants may last up to two years. However, both patients and clinicians are searching for permanent implants for lasting results.

For permanent injectable implant, there are liquid and solid fillers available on the market. Polyacrylamide gel and silicone gel are injectable liquid fillers. Polyacrylamide gel remains pliable and soft after it is injected in the body. However, some bacterial infections within the gel have been reported in the literature. Silicone gel, although chemically well tolerated, becomes encapsulated as a foreign body by a chronic inflammatory reaction. The fibrous tissue surrounding the silicone is avascular and a potential site of infection. A number of late infections, granulomas, and palpable masses have been reported following silicone implantation. In addition, the low molecular weight silicone in the gel can slowly migrate into patients' system and cause problem such as nodules, cellulites, and ulcers in other organs. As a result, bacterial infection and migration are major concerns for liquid permanent fillers.

Many permanent solid or semi-solid types of fillers have been tested or disclosed in the literatures as injectable tissue fillers. They are polytetrafluoroethylene paste, polymethylmethacrylate beads, dextranomer beads, hydrogel beads, metallic particles with carbon coating, carbon particles, silicone particles, ceramic particles, glass beads, etc. They are usually very fine solid particles with a specific gravity higher than water. To avoid clumping and injection difficulty, the particles have to be suspended in a high viscosity carrier and injected subcutaneously through a small needle for both soft and hard tissue augmentation. However, limited success has been reported in some of these approaches. The clinical results were mostly disappointing due to acute or chronic adverse tissue reactions, clumping of particles, injection difficulty, and filler migration to other locations.

With the commonly practiced injection technique, filler particles have to be relatively small to pass through the small bore needle. Fine fragments can be generated in the injection procedure if the fillers are not strong enough to endure the high shear force in the injection. Small and fine particles tend to migrate through the circulatory system and/or be engulfed by the macrophages and move to other undesired sites. For example, undesirable migration and serious granulomatous reactions were reported for polytetrafluoroethylene (PTFE) particles (about 1-100 microns in diameters) suspended in glycerine. It is preferred to have filler particles as larger as possible to avoid the adverse side effects. However, large particles tend to clump and form aggregation in the syringe and inhibit injection.

Polymethylmethacrylate bead (PMMA, Artecoll®) is another solid filler for facial wrinkles and lines correction. The PMMA is formulated into solid microspheres around 32-40 microns in diameter and are suspended in 3.5% collagen solution. After the collagen within the mixture degrades within 2 to 5 months, the solid microspheres are encapsulated by body's own collagen in about 2 to 4 months. This structure adds tissue augmentation without migration of the microspheres. However, the solid beads are relatively heavy. Palpable masses, particles precipitation, clumping and injection difficulty were reported by practitioners. Palpable masses are suspected to be caused by clumping of filler when the carrier is resorbed by the body.

Deformable hydrogel disks address the issue of stiff and palpable masses from solid fillers such as PMMA beads. Hydrogel disks three times larger than the inside diameter of the injection needle were disclosed in U.S. Pat. No. 5,007,940. The outside diameters of the disks are from about 0.005 to 0.2 inch with a lubricious surface. They are flexible and folded when they are forced to pass through the needle, but return to the original disk shape without any damage. They are also lighter in weight and reduce some of the particles precipitation and clumping issues. However, hydrogel is lubricious and known not to adhere to the surrounding tissue, migration of this material to other organs (such as brain tissue) is still a concern.

There are many efforts in trying to resolve the issue of filler clumping and precipitation. The particles are carried by fluids of high viscosities, such as collagen, starch, hydrogel, polysaccharides, and oil to reduce the tendency of clumping and precipitation. However, the high viscosities fluids increase injection difficulty and the chance for adverse incidences. Another approach to minimize issue of clumping and precipitation is to reduce the size of the filler particles. With this approach, the average particles size has to be in a delicate balance between too small (the risk of being engulfed by macrophages and lead to migration) and too big (injection difficulty). The third approach to this clumping problem is to reduce the filler concentration in the composition. However, patients have to be treated multiple times to achieve satisfactory result. Thus, there remains a very important need for a treatment that will provide stable and injectable biocompatible filler. It is desirable to have fillers that have relatively smooth surface and are small enough to be injected through a small bore needle to avoid scar and pain during the procedures. The particles should be large enough so that they won't cause complications such as migration or removal by phagocytes. It will be ideal if the injected fillers are homogenously distributed in the carrier before the injection so that there is no clumping or injection difficult. It is also important for the fillers to remain evenly distributed after the injection to avoid palpable mass after the carrier is resorbed in the body. It is an object of the present invention to provide a novel solution for tissue filler of the human or animal body, giving a long shelf life and minimum side effect.

### SUMMARY OF THE INVENTION

The present invention provides a new composition for treating tissue contour deficiencies, skin defect, urological disorders, gastric fluid reflux disorders, etc., by injecting endoscopically or subcutaneously a biocompatible fluid composition containing a plurality of hollow particulate fillers which are characterized as being stable, biocompatible and non-precipitating. The hollow particulate fillers with a lower “effective density” resolve the precipitation and clumping issue by matching the density of the hollow particles with the carrier. Each of the hollow particulate filler has a least one void inside the particle. If multiple voids exist in one particle, the voids can be either connected or disconnected with each other. The size of the void can be tailored to enable particle with effective density comparable to that of carrier. The material used for the particle is biocompatible and is either biodegradable or nonbiodegradable. The hollow particulate filler is injectable endoscopically or subcutaneously through small bore needles with a biocompatible fluid carrier.

The hollow particulate filler is free of sharp corner or edge. It can be spherical, elliptic, oval, etc. with a smooth non-porous outer surface to avoid inflammation or other adverse body reaction. The average cross sectional dimension ranges from about 20 μm to about 500 μm, preferably, from about 30 μm to about 200 μm. The particulate filler is able to secure itself into the injection position through the large particle size which can not be engulfed by the macrophages in the body. Aggregation and injection difficulty can be minimized by the lower density and the smooth non-tacky particulate surface. After the injection, pluralities of hollow particles in the composition occupy a predetermined volume when the carrier is slowly removed from the body. According to the present invention, the hollow particles have a lower effective density comparable to carrier and are evenly distributed in the body without clumping. Because this homogenous suspension of hollow particles is not affected by the change in viscosity during the resorption of carrier in the body, the hollow particles remain evenly distributed in the body without causing palpable masses at the injection sites.

The hollow particulate filler of the present invention is biocompatible. The biocompatible materials can be polymer, metal, metal oxide, carbon, ceramic, glass, etc. The configuration of the void in the particle is random and can be spherical, elliptic, oval, etc. Multiple voids in each particle are also possible. The void in the particles can be an empty space or filled with air, gas, water, or liquid, etc. Alternatively, the void can be filled with a bioagent. The bioagent is released into the body fluid after it is injected into the body. In another preferred embodiment of the present invention, the particulate fillers comprise radiopaque agent, contrast agent, or mixtures thereof, providing assistance to the operation procedure and detection.

According to the present invention, the carrier mixed with hollow particles can possess a low viscosity without causing precipitation. The biologically compatible carrier cause minimal tissue reaction and is removable or metabolized in the body. Due to this relatively lower viscosity, a larger volume of hollow particles can be used in the composition without injection difficulty or clumping. The hollow particulate fillers are typically present in a concentration from about 10%-80% of total volume of the composition, more typically from about 20% to about 60%. The amount of hollow fillers in the composition varies according to the size of the injection needle and the location of treatment.

The following terms have these meanings as used herein:
1. The term “void” means an empty space completely within the walls of a particle.
2. The term “hollow” means at least one void in a particle.
3. The term “effective density” means the weight of the particle divided by the total volume of the particle including the hollow space within the walls of the particle.

### BRIEF DESCRIPTION OF THE DRAWINGS

FIG. 1 shows a hollow particulate filler in accordance to the present invention.

FIG. 2 shows a cross sectional view of the hollow particle illustrated in FIG. 1 in accordance to the present invention.

FIG. 3 shows a cross sectional view of a hollow particle in accordance to the present invention. The hollow particle has multiple shells with a hollow core.

FIG. 4 shows a cross sectional view of a hollow particle in accordance to the present invention. The hollow particle has multiple voids inside the particle.

FIG. 5 shows a cross sectional view of a hollow particle in accordance to the present invention. The hollow particle has multiple voids inside. Each void is surrounded by a shell.

FIG. 6 shows a cross sectional view of a hollow particle in accordance to the present invention. The hollow particle has multiple voids with a foam or sponge like configuration.

### DETAILED DESCRIPTION OF THE INVENTION

The present invention addresses those aspects of designing an ideal filler composition for tissues that need to be repaired, augmented or strengthened. Other than the treatment of lost skin volume by plastic or reconstructive surgery, tissue fillers can be used to correct aphonia or dysphonia caused by paralysis of the vocal cords, to correct defect or injury, to the augmentation of hypoplastic breast, to the augmentation of scar tissue, to the treatment of urological disorders (e.g. urinary incontinence), to the treatment of incompetent anal sphincters, to the treatment of vesicoureteral reflux, and to the treatment of gastric fluid reflux by endoscopical or subcutaneous injection of biocompatible hollow particulate fillers into the submucosal or dermal tissue. Since the invention is closely related to the augmentation of soft tissue for the treatment of lost skin volume, it will be described in details hereto.

It is typical for injectable particulate fillers to be suspended in a fluid carrier to assist the injection. However, the major issue for this approach is filler clumping and precipitation either before or after injection procedure. The clumping before injection will cause injection difficulty. On the other hand, the clumping after injection may cause palpable masses at the injection sites. There are many efforts in trying to resolve the issue of filler clumping and precipitation without satisfactory result. Thus, there remains a very important need for a treatment that will provide stable and injectable biocompatible filler. It is an object of the present invention to provide a novel solution for tissue filler of the human or animal body, giving a long shelf life without clumping or precipitation.

A simple mathematical equation can be used to explain the phenomena of particle precipitation (or clumping) in the carrier. For a spherical particle of radius R and effective density ρ in a fluid carrier of density σ, there are three forces acting on the particle:

1. The gravity force W acting downwards on the particle is given by

W=4πR³ρg/3

2. The buoyant force U acting upwards is given by

U=4πR³σg/3

3. The dragging force F acting upwards (or downwards depending on the moving direction. It is assumed upward here.) by the fluid carrier is given by

F=6πηav

Where η is viscosity, g is gravity, a is acceleration, v is velocity of the particle. The net downward force, N, is

N=W−(U+F)

N=4πR³ρg/3−(4πR³σg/3+6πηav)

N=4πR³(ρ−σ)g/3−6πηav

The net downward force is responsible for the acceleration of the particle. As the velocity of the particle increases, the dragging force will also increase. At some point, the downward and upward forces acting on the particle are balanced and the net force is zero (N=0).

6πηav=4πR³(π−σ)g/3

v=2R³(ρ−σ)g/9ηa (1)

If the density of particle is the same as that of carrier (ρ=σ), the velocity of the particle, v, would be zero, and the particle will remain in the rest position without precipitation. However, if the density of particle is not the same as that of carrier, the velocity won't be zero and is proportional to the difference in density between the particle and the carrier as shown in Equation 1. A reduced density difference can reduce the velocity of the particle and postpone the precipitation. The direction of the particle movement will depend on the densities of particle and carrier. For particle with a higher density than the carrier, it will move downwards and precipitate eventually. On the other hand, the particle will float to the top if it has a lower density than the carrier. As also indicated in Equation 1, the velocity is reversely proportional to the viscosity. Carrier with a higher viscosity, η, will reduce the particle velocity and slow down the movement. However, the movement can't be stopped as long as there is a density difference, and the particle will precipitate eventually.

Currently, all the injectable fillers on the market (polytetrafluoroethylene, carbon, calcium hydroxyapatite, polymethylmethacrylate, poly lactic acid) have densities higher than 1.2 g/cm³ (1.2 g/cm³ for PMMA, 1.2 g/cm³ for PTFE, 1.25 g/cm³ for PLA, 3.1 g/cm³ for HA, 1.5 g/cm³ for PGA). Physiologically acceptable fluids such as water, saline (˜1 g/cm³ for both) are common carriers used with those fillers. They are usually mixed with suspension agents such as collagen, methylcellulose (MC), carboxymethylcellulose (CMC) for increased viscosity. The resulting fluids have densities usually less than 1 g/cm³. For example, the solid PMMA particle in Artecoll™ has a density of 1.2 g/cm³, and density of the 3.5% collagen carrier is 1.04 g/cm³. As a result, the downward velocity of the particle is positive and needs to be slowed down by the higher viscosity of the suspension agent. However, a small bore needle is preferred by the practitioners for less pain and scar on the patient to be treated, the higher viscosity cause injection difficulty during the procedures. As what discussed above, the solid PMMA particle still will precipitate eventually even with a thick carrier.

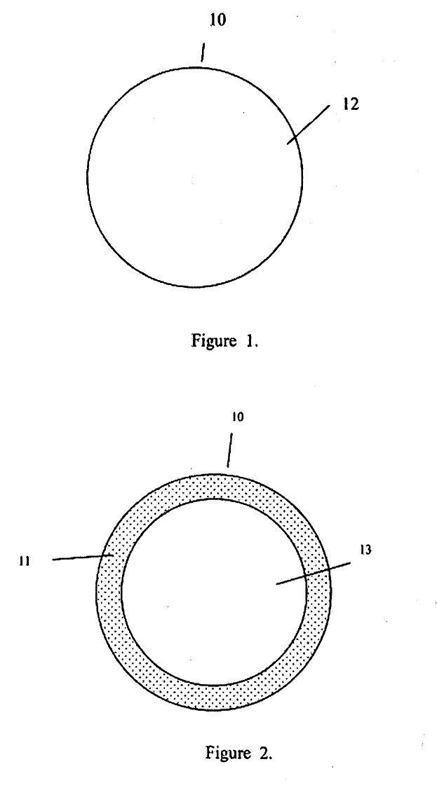

The goal of this invention is to present a new filler material to resolve the precipitation and clumping issue. In this invention, instead of solid particle currently used in the market, hollow particle with a lower “effective density” is used as filler. The intention is to use void to reduce the effective density of particle to avoid precipitation due to the density difference between the filler and the carrier. An example of the hollow particle is illustrated in FIGS. 1 and 2. Its outer diameter is R and inner diameter is r. The void inside the particle is either an empty space or filled with air or gas. The density of the gas is insignificant and ignored in this calculation for simplification. As what was described above, the effective density of the particle is preferred to be comparable to the density of the carrier to avoid precipitation. Then, the radius of the void required to “lighten” the particle can be calculated as following:

σ=ρ

σ=ρ=ρ′(4πR³/3−4πr³/3)/(4πR³/3)

σ=ρ=ρ′(R³−r³)/R³

r=R(1−σ/ρ′)^{1/3} (2)

Where ρ is the effective density of the particle, p′ is the density of the shell material, σ is density of the carrier. Equation 2 indicates that the effective density of the particle, ρ, can be reduced by a void inside the particle, and precipitation can be avoided even with a high shell material density, ρ′. Similarly, the portion of the hollow space, P, in the particle required to reduce the effective density can be calculated as

P=Sv/Sp=(4πr³/3)/(4πR³/3)

P=(r/R)³

P=(1−σ/ρ′)

Where Sv is the volume of void, Sp is the total volume of particle. To simplify the equation, the density of the gas in the void is small enough and ignored in this calculation. Again, the hollow PMMA particle illustrated in FIGS. 1 and 2 is used as an example. PMMA has a density of 1.2 g/cm³, and that of the 3.5% collagen carrier is 1.04 g/cm³. Assuming the outer radius of hollow PMMA particle is 15 microns, the radius and the size of the void inside the particle to avoid precipitation can be calculated as the following:

r=15(1−1.04/1.2)^{1/3}

r=7.66 microns

P=(1−1.04/1.2)

P=0.13=13%

When the radius of the void is 7.66 microns and 13% of the particle's total volume is void, the hollow particle's effective density will be comparable to that of the carrier which is 1.04 g/cm³. As a result, the hollow PMMA particle can be suspended in the 3.5% collagen carrier without precipitation. Furthermore, if a saline solution (density ˜1 g/cm³) is used as the carrier for hollow PMMA particle, the radius and size of the void inside the particle to avoid precipitation can be calculated as the following:

r=15(1−1/1.2)^{1/3}

r=8.25 microns

P=(1−1/1.2)

P=0.17=17%

With a low density carrier, a larger void (8.25 microns in radius, 17% of the particle's total volume) in the particle will be needed to reduce its effective density to match the density of carrier. The benefits for the low density carrier are a lower viscosity fluid without injection difficulty, and potentially a higher load of filler particles in the composition.

Another embodiment of this invention comprises hollow particulate fillers with voids partially or totally filled with a liquid. The voids in the hollow particle can be an empty space by vacuum in the fabrication of the particles. Alternatively, a liquid with a lower density than the shell material can lower the effective density of the particle to avoid precipitation. Suitable liquids for this invention are any physiologically compatible liquids such as water, PBS and saline. The liquid or gas in the void can be introduced during the synthesis or fabrication process of the hollow particle. Alternatively, the gas or liquid can be introduced into the particle by high pressure, centrifuge, diffusion, etc. With those techniques, it will be convenient to control the amount of gas or liquid in the void in order to adjust the effective density of the hollow particle. According to this invention, a hollow particle with a large void can be made, and its effective density can be fine-tuned by introducing appropriate amount of low density gas or liquid into the void.

Hollow particles have been used as pigment, drug delivery carrier, protecting agent, adhesive, and texturing agent for cosmetics, etc. The hollow particulate filler of the present invention is biocompatible and capable of homogenously suspending in water or other low viscosity carrier. Many biocompatible materials can be used in this invention. They can be polymer, metal, metal oxide, carbon, ceramic, glass, etc. The configuration of the hollow particle is random and can be spherical, elliptic, oval, etc. Its outer surface should be smooth without pore or sharp corner to avoid inflammation or other adverse body reaction. The configuration of the void in the particle is random and can be spherical, elliptic, oval, etc. Multiple voids in each particle are also possible. The voids can be isolated or interconnected to form porous mesh, foam, or sponge. These various void configurations can be controlled by the materials, agents, surfactant, and processing parameters introduced during the fabrication of the hollow particulate filler. For polymeric hollow particle, the shell thickness is controlled by the length of polymerization during the fabrication process and provides hollow particles with various strength and “effective density”. Longer polymerization time can produce polymer with longer chains and thus, shell thickness. As a consequence, the void is smaller with a higher “effective density”.

There are many methods to produce hollow particles. They are solvent evaporation, emulsion polymerization, interfacial polymerization, phase separation, heat expansion, and density separation, etc. For polymeric hollow particle, a polymer shell was formed over a soluble substrate of silica, mica, alumina, etc. as disclosed in US 2004/0219360A1. The surface of the substrate has hydroxyl groups and is able to initiate living radical polymerization. The substrate particles are suspended in a solvent with monomers. After the polymerization is initiated by the each substrate surface and an appropriate shell thickness is achieved, the substrate is then dissolved in an etching agent to form a hollow particle. A relatively uniform shell thickness can be achieved with this method. The size of the void is controlled by size of the substrate and the amount of crosslinking agent used in the polymerization. An alternative method to make hollow particles was also disclosed in the same patent application. The silica substrate is assembled with polymeric nanospheres in a solution. The assembled composite is subsequently heated to a temperature above Tg of the nanospheres allowing the polymer to flow over the substrate and resulting a uniform shell. The substrate can be removed with the etching agent as described before. Japanese Patent JP2003181274A2 describes a method for manufacturing hollow polymer particles with emulsion. Oil droplets containing monomers and an organic solvent form a shell layer after polymerization. Then the particles are made hollow by removing the organic solvent. U.S. Pat. No. 4,594,363 disclosed an emulsion polymerized carboxylated core polymer with a polymer shell. The expansion of the carboxylated polymer core with a base produces voids in the particles. U.S. Pat. No. 4,972,000 disclosed a method to form hollow particles by the difference in density between the monomer and its polymer during the polymerization. Canadian patent 888,129 disclosed the use of blowing agent in the polymer particles to form voids in the particles. EP0462388A3 describes a method to manufacture hollow particles having an average particle diameter of 0.1-30 microns and a shell thickness of 0.01-4 microns. The volume ratio of the internal void to the total volume in the hollow particles is 40-80%. Japanese patent application JP2002105104A2 describes a process to produce hollow polymeric particles. A mixture of monomer and cross-linking monomer are mixed with an oily substance through a microporous membrane into an immiscible liquid and producing an emulsion comprising both dispersed and continuous phases. After the polymerization, the monomer forms the solid polymer shell having an inner core with oily substance. The hollow particles are further produced by removing the oily substance in the solid polymer particles. U.S. Pat. No. 4,133,854 disclosed a method to product glass, metal or plastic hollow spheres. A blowing agent was mixed with particles and exposed to high temperature to decompose the agent and expand the particles. U.S. Pat. No. 4,782,097 disclosed a method to create polymer or carbon hollow particles by a blowing agent which decomposes at high temperature. U.S. Pat. No. 4,968,562 described a two-step water-in-oil-in-water emulsification polymerization process to prepare hollow polymeric particle. A majority of particles have multiple interior voids. U.S. Pat. No. 4,257,799 disclosed a method to produce glass hollow particles having an outside diameter from about 100 microns to about 500 microns. U.S. Pat. No. 3,030,215 described method to produce alkali metal silicate based glass hollow particles with an outside diameter from about 5 microns to about 5000 microns. U.S. Pat. No. 6,136,891 described a method to produce hollow particles with oxides of aluminum, silicon, zirconium and/or transition metal. The disclosures of each of those patents are incorporated herein by reference in their entirety. Suitable procedures described in those disclosures may be employed or modified to prepare hollow particle within the scope of this invention.

Many hollow particles with various materials and sizes are commercially available. For example, poly(methylmethacrylate) particles are sold by Sensient Technology under the name “Covabead”. Terpolymer particles of vinylidene chloride, acrylonitrile and methyl methacrylate are sold by Nobel, Sweden, under the name “Expancel”. Soda-lime borosilicate glass hollow particles are sold by 3M Corp. under the code “D32/4500” and “B46/4000”. It is preferred to use one of those commercially available hollow particles in this invention.

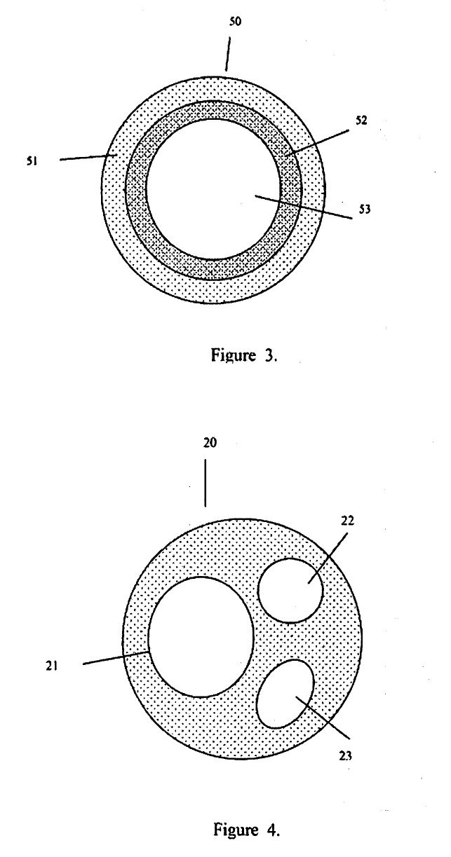

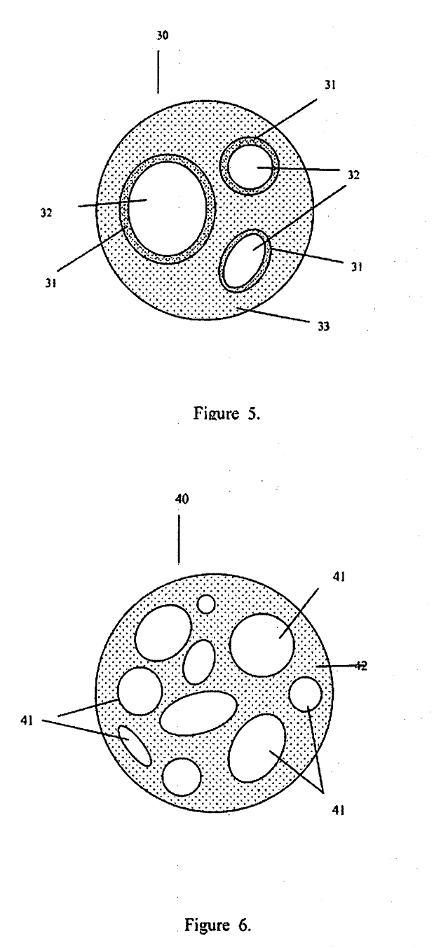

A preferred embodiment of this new composition according to this invention comprises a plurality of injectable hollow particulate fillers suspended in a biocompatible carrier. Each hollow particle has a shell of biocompatible material and a hollow interior. Each hollow particle 10 described here has a smooth surface 12 without sharp corner and edge as shown in FIG. 1. The void in the particle has an average volume from about 0.1% to about 74% of the total particulate volume as shown in the cross sectional view of particle 10 in FIG. 2. The wall thickness 11 of particle 10 is from about 0.1% to about 98% of the particulate cross sectional dimension. The shape of the void 13 is random. It can be spherical, oval, etc. Alternatively, there can be more than one layer of shell in the wall 51, 52 of the particle 50 as shown in FIG. 3. Each layer can be made by either the same material or a different material. The spherical void 53 is near the core of the particle 50. Alternatively, there are multiple voids 21-23 in each particle 20 as shown in the cross-sectional view illustrated in FIG. 4. Depending on the fabrication process, there could be a second wall 31 surrounding each void 32 as shown in the cross section of particle 30 as illustrated in FIG. 5. The material used for the second wall 31 can be the same material used in the main wall 33 or a different material. FIG. 6 illustrates the cross section of hollow particle 40 having foam or sponge-like voids 41 inside the particle 40. The voids 41 are either interconnected or separated from each other depending on the fabrication processes. The types of void described here are controlled by the amount of blowing agent, material, surfactant, and the processing method in making the hollow particle.

According to the present invention, the hollow particle with a density comparable to carrier will also avoid clumping and palpable masses at the injection sites. After the carrier is injected and resorbed in the body, the size of the hollow particle provides fixation at the injection location and prevents the undesirable migration to other parts of the patients' body. It is obvious that large particles, especially those larger than 20 microns, are less likely to be engulfed by microphage or other elements in the body. The preferred average diameters of the hollow particles range from about 20 microns to about 500 microns, more preferably between about 30 and 200 microns. However, clumping of the particles may occur before the carrier is totally resorbed and cause palpable mass. It is suspected that the body temperature reduces the carrier viscosity and thus accelerates the precipitation of the particles. According to this invention, the hollow particles have effective density comparable to the carrier and suspend evenly in the carrier. As indicated in Equation 1, the suspension of hollow particle is not affected by change in viscosity during the resorption of carrier in the body. The hollow particles remain at the injection site without clumping or forming palpable masses.

According to the present invention, the carrier mixed with hollow particles can possess a low viscosity without causing precipitation. Due to this relatively lower viscosity, a larger volume of particles can be used in the composition without injection difficulty or clumping. The ability to incorporate a larger volume of particles in the composition is advantageous because the undesirable “over-correction” or multiple injections can be minimized. The “over-correction” means that the physicians need to “guess” and inject more solution in the patients to compensate for the loss in carrier volume later on. This uncertainty can be minimized with a higher percentage of fillers in the composition. The hollow particulate filler is typically present in a concentration of from about 10-80% of total volume of the composition, more typically from about 20% to about 60%. The amount of hollow filler changes according to size of the injection needle, and the type and location of treatment.

The critical requirement for the hollow particle is that the material used should be biocompatible with a minimum inflammatory reaction. The material can be degradable or non-degradable by the body fluids or the action of tissue enzymes. The suitable non-degradable materials are silicone, polysiloxane rubber, polydimethylsiloxane, polyurethane, polytetrafluoroethylene (PTFE), glass, ceramic, metal, carbon, calcium hydroxyapatite, polymethylmethacrylate, polymethacrylate, acrylic polymer, polybutylmethacrylate, polyethylene imine, polyethylene terephthalate (PET), polyesters, polybutester, polyacrylonitrile, polyaryletherketone, PEEK, polyethylene, polypropylene, ethylene propylene copolymer, polyolefins, fluorinated ethylene propylene copolymer, polyethylene vinyl acetate, sodium acrylate polymer, polycarbonates, polyamides, polyamideimides, polyimides, polyaryletherketones, polytetramethylene oxide, polysulfones, polyphenylenesulfides, polyhydroxy ethyl acrylate, polyhydroxy ethyl methacrylate, polyacrylamide, polyacrylamide copolymer, sodium acrylate and vinyl alcohol copolymer, polyvinyl alcohol, polyacrylic acid, polymethylacrylic acid, polyacetals, polyvinyl acetate and acrylic acid ester copolymer, polyvinyl pyrrolidone, polyethylene glycol, polypropylene glycol, polyvinyl acetate, polyvinyl acetate and methyl maleate copolymer, polyarylethemitriles and aromatic polyhydroxyethers, Hypan, poly(2-hydroxyethyl methacrylate)(polyHEMA), polystyrene, isobutylene-maleic anhydride copolymer, polyethylene oxide, polyvinylidene or copolymer or mixtures thereof. Those skilled in the art will recognize the various biostable materials that may be used to fabricate the particles. The preferred materials are PMMA, PTFE, PET, polymethacrylate, and silicone. According to this invention, PMMA is the preferred material used for non-degradable hollow particle because its ability to stimulate tissue growth surrounding the PMMA particle. If more than one material is used, PMMA should be used as part of, or the whole, outer shell which is in contact with body fluid.

A variety of biodegrabale materials can be used in the hollow particle. They are polyglactin, poliglecaprone, lactomer, polycaprolactone, poly(dioxanone), poly(glycolide-co-trimethylene carbonate), polytrimethylene carbonate, poly(glycolide-co-trimethylene carbonate-co-dioxanone), polyhydroxyalkanoate, polyhydroxybutyrates, polyhydroxyvalerates, polyalkylene oxalates, polyalkylene succinates, poly methyl vinyl ether, poly maleic anhydride, chitin, chitosan, poly(ε-decaloactone), poly malic acid, poly amino acids, polyphosphazenes, polyphosphoesters, polyamides, poly iminocarbonates, polycarbonates, polyorthocarbonates, polyethylene carbonate, polydioxanone, polyketals, proteinaceous polymers, polyesters, polyester amides, polysaccharides, starch, poly lactic acid, poly glycolic acid, or combination or copolymer thereof. Other than the materials described above, certain types of surface erosion materials are also suitable for this application. They are hydrophobic, but the chemical bonds of the polymers are highly susceptible to hydrolysis. As a result, water penetrates slower than the conversion rate of the polymers into soluble materials. Surface erosion results in the thinning of the material over time while maintaining its bulk integrity. This type of polymer is known as surface erosion or bioerosion material. The examples this type of material are polyanhydrides, methyl vinyl ether maleic anhydride copolymer, and polyorthoesters. Those skilled in the art will recognize the various biodegradable materials that may be used to fabricate hollow particles.

According to this invention, a variety of biocompatible carriers can be used to suspend the hollow particles to avoid clumping before and after injection. Many physiological solutions such as saline, water, PBS solution can be used as carrier. Alternatively, it can be formulated by mixing with a thickening agent or a suspension agent to modify the viscosity to provide the composition with comparable density with the hollow particle for the homogenous particulate suspension. The choice of suitable carrier will depend on the particle size, the amount of fillers, the size of injection needle and the nature of the fillers. The suitable thickening or suspension agent includes all the biocompatible agent known in the art to act as thickening or suspension agent. Some typical thickening or suspension agents are Acacia, Carbomer copolymer and homopolymer, Carbomer interpolymer, hydrogel, polysaccharide, macrocyclic polycsaccharide, oligosaccharide, starch, acetyl starch, cellulose, cellulose derivatives, methylcellulose, carboxymethylcellulose sodium, carboxymethylcellulose (CMC), ethyl (hydroxyethyl) cellulose (EHEC), ethylcellulose, hydroxypropyl cellulose, hydroxypropyl methylcellulose (HPMC), ethylcellulose, alkyl cellulose, alkoxy cellulose, hydroxy ethyl cellulose, copovidone, povidone, gelatin, glucose, Guar gum, hypromellose, hypromellose acetate succinate, maltodextrin, syrup, agar, alamic acid, aluminum monostearate, attapulgite, gellan gum, hypromellose, maltodextrin, pectin, propylene glycol alginate, sodium alginate, calcium alginate, colloidal silicon dioxide, tragacanth, xanthan gum, lecithin, tridobenzene derivatives, iohexyl, iopamidol, iopentol, sucrose, carrageenan, agarose, mannitol, saccharin sodium, sorbitol, cephalin, acetylenic diol, Carbowax, polyorgano sulfonic acid, alkoxylated surfactants, alkylphenol ethoxylates, ethoxylated fatty acids, alcohol ethoxylates, alcohol alkoxylates, polyethylene oxide, poly(propylene oxide), poly(ethylene glycol), poly(propylene glycol), poly vinyl alcohol (PVA) polymer or copolymer, polyacrylamine, poly(vinylcarboxylic acid), polymethacrylic acid, polyacrylic acid polymer or copolymer, poly amino acids, albumin, collagen, fibrin, bioglue, cellulosics, Carbopol, Poloxamer, Pluronic, Tetronics, PEO-PPO-PEO triblocks copolymer, Tetrafunctional block copolymer of PEO-PPO condensed with ethylenadiamine, polyHEMA polymer or copolymer, Hypan polymer or copolymer, starch glycolate polymer or copolymer salt, polyoxyalkylene ether, polyvinyl pyridine, polylysine, polyarginine, poly aspartic acid and poly glutamic acid, polytetramethylene oxide, poly(hydroxy ethyl acrylate), poly(hydroxy ethyl methacrylate), methoxylated pectin gels, cellulose acetate phthalate, organic oils, B-glucan, polysorbate, lactic acid ester, caproic acid ester, hyaluronic acid, dextrin, dextran, dextrose, and mixture of the above. Poloxamers, Pluronics, CMC, HPMC, gelatins, collagen, hyaluronic acid, and acetyl starch are preferred because they are readily and economically available and are easy to work with. The patient's own plasma can also be used as a carrier. It may be derived from blood withdrawn from the patient, centrifuged to remove cells (or not) and mixed with appropriate amount of fillers to form an injectable composition. The thickening agent is typically present in a concentration of from about 0.0-40% of the total weight in the carrier, more typically from about 0.1% to about 20%.

Alternatively, a radiopaque agent can be introduced in the hollow particle for enhanced visibility under fluoroscopy. The radiopaque agent can be barium sulfate, silver, gold, tantalum, etc. If barium sulfate is used, sufficient amount of barium sulfate powder can be blended with the material used for the shell during the fabrication of the hollow particle. As a result, all of the barium sulfate is attached to the fillers without free particles of barium sulfate in the composition. It is also feasible to place radiopaque agent inside the void.

Alternatively, a bioactive ingredient can be embedded in the hollow particle to promote cell proliferation, connective tissue response, and the interaction between the filler particle and the cells to enhance the bonding between the filler and surrounding tissue. The bioactive ingredient can be growth factors, hormones, cytokines, bactericidal agents, antimicrobial agents, antiviral agents, cell adhesion promoter, Vitamin C, drug or other pharmacologically active compounds. The bioactive ingredient can be introduced into the void during the fabrication of the hollow particle or by diffusion after the particle is made. It becomes part of the filler particle and released through the shell or when the particle degrades in body fluids. Alternatively, bioactive ingredient can be blended with the shell material during the fabrication and released by diffusing out of the filler particle. Compared with the fragile coating on the particulate surface in other methods, the bioactive ingredient in this invention has the advantage of being part of the hollow particle with stronger fixation to endure the injection force and only be released when it is in the body.

The disclosed composition in this invention normally contains a major amount of water (preferably purified water, physiological saline, or the like) in addition to the fillers and thickener. The compositions can also be lyophilized for longer shelf life. Minor amounts of other ingredients such as anesthetic agent and preservative may also be present depending upon the route of administration and the preparation desired. The compositions can also be isotonic (i.e., it can have the same osmotic pressure as body fluids).

An aspect of the present invention encompasses an anesthetic to decrease the pain or discomfort associated with injection of the composition. Example of anesthetics include but are not limited to lidocaine, xylocaln, novocain, benzocain, prilocaln, ripivacain, propofol, benzyl alcohol, and chlorobutanol. Typically the anesthetic will be used with aqueous base and thus will be mixed with the composition prior to administration. A suitable concentration of the anesthetic will be from 0.01% to 6% based on the total weight and the agent selected.

Alternatively, isotonicity of this invention may be accomplished using sodium chloride, or other pharmaceutically acceptable agents such as dextrose, boric acid, sodium tartrate, propylene glycol or other inorganic or organic solutes. A pharmaceutically acceptable preservative can be employed to increase the shelf-life of the compositions. Benzyl alcohol may be suitable, although a variety of preservatives including, for example, parabens, thimerosal, chlorobutanol, or benzalkonium chloride may also be employed. A suitable concentration of the preservative will be from 0.02% to 2% based on the total weight and the agent selected.

According to the present invention, the injectable hollow particulate fillers/carrier composition disclosed herein can be in a ready for use pre-filled sterile syringe with both filler and the biocompatible carrier. Or, it can be provided in a vial in the form of sterilized dry fillers. In this embodiment, the end user could add carrier, water or other pharmaceutically acceptable carrier and/or additional additives for preparation of suspension prior to injection. Alternatively it can be in a two pre-filled syringes, wherein one syringe contains dry and sterilized fillers and the other syringe contains a pharmaceutically acceptable carrier solution. The dry fillers and the carrier are ready to be mixed for injection by pushing the composition back and forth in the syringes or mixed in a separate container until a homogenous suspension is reached. The compound disclosed herein may be optionally be sterilized by Gamma or E-beam irradiation, filtering, heating or exposure to ethylene oxide gas. Once the fillers/carrier composition has been prepared by any one of the existing processes, it can be applied by subcutaneous or endoscopical injection into the patient to be treated. For the augmentation of the dermal tissue, the injection of the present invention can be carried out by using syringe with needle of from 18 gauge to 30 gauge. The size of the needle will be determined by the filler composition, the depth of the injection site, the injection volume, etc. The composition is then injected through the needle into patient's body. The hollow particulate fillers can't be digested or eliminated by macrophage or other elements of immune system.

According to the present invention, a preferred method for the augmentation of dermal tissue is to inject the composition subcutaneously into layer of the skin at the treatment site. The present invention also provides method of treating GERD by administering the injectable hollow particulate fillers/carrier composition through a needle to the sphincter wall near esophagus endoscopically or laparoscopically. The narrower esophageal sphincter allows easier muscle contraction and prevents the regurgitation of the gastric fluid into the esophagus. Some cases of urinary incontinence occur when the resistance to urine flow has decreased excessively. Continence is restored by injecting the present invention into the urethra tissue near the urethra sphincter to reduce the ureters lumen and increase resistance to urine outflow from the bladder. For patients with vesicoureteral reflux, it can be treated by injection of the present invention into patients' ureteral tissue. This invention can also be used to repair fecal incontinence or defective anal sphincter muscles by administering an effective amount of injectable hollow fillers into the defect or anal sinuses.

Various modifications of the invention described herein will be apparent to those skilled in the art. Such modifications are also intended to fall within the scope of this invention.

1. A biocompatible injectable tissue implant composition comprising hollow particles suspended in a biocompatible carrier, said hollow particles having smooth, non-tacky and non-porous outer surfaces, wherein the voids of the said hollow particles comprise from about 0.1% to about 74% of the total particulate volume, said hollow particles having an average cross sectional dimension from about 20 to about 500 microns.

2. The injectable tissue implant composition of claim 1, wherein said hollow particles is selected from the group consisting of natural polymer, synthetic polymer, metal, metal oxide, glass, carbon, ceramic, degradable material, non-degradable material, or combination thereof.

3. The injectable tissue implant composition of claim 1, wherein said hollow particles comprise polymethylmethacrylate or its copolymer in the outer shell.

4. The injectable tissue implant composition of claim 1, wherein the effective density of said hollow particles is sufficient low allowing even suspension in the said carrier.

5. The injectable tissue implant composition of claim 1, wherein said void is an empty space or comprises a gas or a liquid.

6. The injectable tissue implant composition of claim 1, wherein said void is an empty space or comprises water.

7. The injectable tissue implant composition of claim 2, wherein said degradable material is selected from the group consisting of polyglactin, poliglecaprone, lactomer, polycaprolactone, poly(dioxanone), poly(glycolide-co-trimethylene carbonate), polytrimethylene carbonate, poly(glycolide-co-trimethylene carbonate-co-dioxanone), polyhydroxyalkanoate, polyhydroxybutyrates, polyhydroxyvalerates, polyalkylene oxalates, polyalkylene succinates, poly methyl vinyl ether, poly maleic anhydride, chitin, chitosan, poly(ε-decaloactone), poly malic acid, poly amino acids, polyphosphazenes, polyphosphoesters, polyamides, poly iminocarbonates, polycarbonates, polyorthocarbonates, polyethylene carbonate, polydioxanone, polyketals, proteinaceous polymers, polyesters, polyester amides, polysaccharides, starch, poly lactic acid, poly glycolic acid, polyanhydrides, methyl vinyl ether maleic anhydride copolymer, polyorthoesters, or combination or copolymer thereof.

8. The injectable tissue implant composition of claim 2, wherein said degradable material is selected from the group consisting of poly lactic acid, poly glycolic acid, or combination or copolymer thereof.

9. The injectable tissue implant composition of claim 2, wherein said non-degradable material is selected from the group consisting of silicone, polysiloxane rubber, polydimethylsiloxane, polyurethane, polytetrafluoroethylene (PTFE), glass, ceramic, metal, carbon, polymethylmethacrylate, polymethacrylate, acrylic polymer, polybutylmethacrylate, polyethylene imine, polyethylene terephthalate (PET), polyesters, polybutester, polyacrylonitrile, polyaryletherketone, PEEK, polyethylene, polypropylene, ethylene propylene copolymer, polyolefins, fluorinated ethylene propylene copolymer, polyethylene vinyl acetate, sodium acrylate polymer, polycarbonates, polyamides, polyamideimides, polyimides, polyaryletherketones, polytetramethylene oxide, polysulfones, polyphenylenesulfides, polyhydroxy ethyl acrylate, polyhydroxy ethyl methacrylate, polyacrylamide, polyacrylamide copolymer, sodium acrylate and vinyl alcohol copolymer, polyvinyl alcohol, polyacrylic acid, polyacetals, polyvinyl acetate and acrylic acid ester copolymer, polyvinyl pyrrolidone, polyethylene glycol, polypropylene glycol, polyvinyl acetate, polyvinyl acetate and methyl maleate copolymer, polyarylethernitriles and aromatic polyhydroxyethers, Hypan, poly(2-hydroxyethyl methacrylate)(polyHEMA), polystyrene, polymethylacrylic acid, isobutylene-maleic anhydride copolymer, polyethylene oxide, polyvinylidene, or copolymer or mixtures thereof.

10. The injectable tissue implant composition of claim 1, wherein said carrier is selected from the group consisting of saline, water, PBS solution, alcohols, or other physiological solutions.

11. The injectable tissue implant composition of claim 10, wherein said carrier further comprising a thickening or suspending agent selected from the group consisting of Acacia, Carbomer copolymer and homopolymer, Carbomer interpolymer, hydrogel, polysaccharide, macrocyclic polycsaccharide, oligosaccharide, starch, acetyl starch, cellulose, cellulose derivatives, methylcellulose, carboxymethylcellulose sodium, carboxymethylcellulose (CMC), ethyl (hydroxyethyl) cellulose (EHEC), ethylcellulose, hydroxypropyl cellulose, hydroxypropyl methylcellulose (HPMC), ethylcellulose, alkyl cellulose, alkoxy cellulose, hydroxy ethyl cellulose, copovidone, povidone, gelatin, glucose, Guar gum, hypromellose, hypromellose acetate succinate, maltodextrin, syrup, agar, alamic acid, aluminum monostearate, attapulgite, gellan gum, hypromellose, maltodextrin, pectin, propylene glycol alginate, sodium alginate, calcium alginate, colloidal silicon dioxide, tragacanth, xanthan gum, lecithin, tridobenzene derivatives, iohexyl, iopamidol, iopentol, sucrose, carrageenan, agarose, mannitol, saccharin sodium, sorbitol, cephalin, acetylenic diol, Carbowax, polyorgano sulfonic acid, alkoxylated surfactants, alkylphenol ethoxylates, ethoxylated fatty acids, alcohol ethoxylates, alcohol alkoxylates, polyethylene oxide, poly(propylene oxide), poly(ethylene glycol), poly(propylene glycol), poly vinyl alcohol (PVA) polymer or copolymer, polyacrylamine, poly(vinylcarboxylic acid), polymethacrylic acid, polyacrylic acid polymer or copolymer, poly amino acids, albumin, collagen, fibrin, bioglue, cellulosics, Carbopol, Poloxamer, Pluronic, Tetronics, PEO-PPO-PEO triblocks copolymer, Tetrafunctional block copolymer of PEO-PPO condensed with ethylenadiamine, polyHEMA polymer or copolymer, Hypan polymer or copolymer, starch glycolate polymer or copolymer salt, polyoxyalkylene ether, polyvinyl pyridine, polylysine, polyarginine, poly aspartic acid and poly glutamic acid, polytetramethylene oxide, poly(hydroxy ethyl acrylate), poly(hydroxy ethyl methacrylate), methoxylated pectin gels, cellulose acetate phthalate, organic oils, B-glucan, polysorbate, lactic acid ester, caproic acid ester, hyaluronic acid, dextrin, dextran, dextrose, or mixtures thereof.

12. The injectable tissue implant composition of claim 1, wherein said composition contains hollow particles in an amount from about 10% to approximately 80% of the total composition weight.

13. The injectable tissue implant composition of claim 1, wherein said hollow particles further comprising radiopaque agent, contrast agent, bioactive ingredient, pharmaceutics, or mixture thereof.

14. The injectable tissue implant composition of claim 1 further comprising anesthetic, preservative, or mixture thereof.

15. A biocompatible injectable tissue implant composition comprising hollow particles suspended in a biocompatible carrier, said hollow particles having a void comprising a volume from about 0.1% to about 74% of the total particulate volume, said void being an empty space or comprising a liquid, said hollow particles having smooth non-tacky and non-porous outer surfaces, said hollow particles having an average cross sectional dimension from about 20 to about 500 microns.

16. The injectable tissue implant composition of claim 15, wherein said hollow particles is selected from the group consisting of natural polymer, synthetic polymer, metal, metal oxide, glass, carbon, ceramic, degradable material, non-degradable material, or combination thereof.

17. The injectable tissue implant composition of claim 15, wherein said hollow particles comprise polymethylmethacrylate or its copolymer in the outer shell.

18. The injectable tissue implant composition of claim 15, wherein said liquid is water or other physiological solutions.

19. The injectable tissue implant composition of claim 15, wherein said carrier is selected from the group consisting of water, alcohol, saline, Pluronics, CMC, HPMC, gelatins, starch, hydrogel, polysaccharide, collagen, hyaluronic acid, or mixtures thereof.

20. A biocompatible injectable tissue implant composition comprising hollow particles suspended in a biocompatible carrier, said hollow particles comprising polymethylmethacrylate or its copolymer in the outer shell, said hollow particles having a void comprising a volume from about 0.1% to about 74% of the total particulate volume, said void being an empty space or comprising water or other physiological fluid, said hollow particles having smooth non-tacky and non-porous outer surfaces, said hollow particles having an average cross sectional dimension from about 20 to about 500 microns,

21. The injectable tissue implant composition of claim 20, wherein said carrier is selected from the group consisting of water, alcohol, saline, Pluronics, CMC, HPMC, gelatins, starch, hydrogel, polysaccharide, collagen, hyaluronic acid, or mixtures thereof.
